SEQUENCE LISTING (176 sequences)

SEQ ID NO: 1 - DNA, 30 bp, Artificial Sequence
  Primer
tcttacttagaataggtcgtcgattcagca

SEQ ID NO: 2 - DNA, 24 bp, Artificial Sequence
  Primer
ccactgatttctgccgcttccgtt

SEQ ID NO: 3 - DNA, 26 bp, Artificial Sequence
  Primer
acacattccaacctgcttgaatacca

SEQ ID NO: 4 - DNA, 24 bp, Artificial Sequence
  Primer
tcttccgcccccttccacaactat

SEQ ID NO: 5 - DNA, 24 bp, Artificial Sequence
  Primer
gagacattcctccgctttcaggcg

SEQ ID NO: 6 - DNA, 24 bp, Artificial Sequence
  Primer
tggaaccgctaaggaaagggggtc

SEQ ID NO: 7 - DNA, 24 bp, Artificial Sequence
  Primer
aacggcattcccgtagcaattggg

SEQ ID NO: 8 - DNA, 24 bp, Artificial Sequence
  Primer
ggatgagattgggtcccagcggat

SEQ ID NO: 9 - DNA, 24 bp, Artificial Sequence
  Primer
tttccaccacgacgtgcatttcgt

SEQ ID NO: 10 - DNA, 24 bp, Artificial Sequence
  Primer
tacaaattgcggggcgtatcgacg

SEQ ID NO: 11 - DNA, 24 bp, Artificial Sequence
  Primer
tcggaagaaaggtgggatccggac

SEQ ID NO: 12 - DNA, 24 bp, Artificial Sequence
  Primer
tggtatggggtcttatcgaagcgc

SEQ ID NO: 13 - DNA, 20 bp, Artificial Sequence
  Primer
ccccgcccagtagtgcctct

SEQ ID NO: 14 - DNA, 20 bp, Artificial Sequence
  Primer
ccgcgggcatcgcgataagt

SEQ ID NO: 15 - DNA, 21 bp, Artificial Sequence
  Primer
cggccatcctggtcctcagga

SEQ ID NO: 16 - DNA, 20 bp, Artificial Sequence
  Primer
tggggactcgcacgaggagg

SEQ ID NO: 17 - DNA, 20 bp, Artificial Sequence
  Primer
ggcaggagcgcaacgacctt

SEQ ID NO: 18 - DNA, 20 bp, Artificial Sequence
  Primer
agtcgggttgctcacgcagc

SEQ ID NO: 19 - DNA, 20 bp, Artificial Sequence
  Primer
tggtgtgcttcctgctcgcg

SEQ ID NO: 20 - DNA, 20 bp, Artificial Sequence
  Primer
tttctccgtgcccgttccgc

SEQ ID NO: 21 - DNA, 20 bp, Artificial Sequence
  Primer
aggtgcccgtagtaggccgg

SEQ ID NO: 22 - DNA, 20 bp, Artificial Sequence
  Primer
ttgggcttggctctgctcgc

SEQ ID NO: 23 - DNA, 20 bp, Artificial Sequence
  Primer
cacgactccccctctccccg

SEQ ID NO: 24 - DNA, 20 bp, Artificial Sequence
  Primer
tgcccgattccccgacccat

SEQ ID NO: 25 - DNA, 20 bp, Artificial Sequence
  Primer
acgggaattgaacccgcgca

SEQ ID NO: 26 - DNA, 19 bp, Artificial Sequence
  Primer
cgggttgctaactcaacgg

SEQ ID NO: 27 - DNA, 24 bp, Artificial Sequence
  Primer
tcaaatgatacatagtgcgataca

SEQ ID NO: 28 - DNA, 18 bp, Artificial Sequence
  Primer
cgaatccctctctttccg

SEQ ID NO: 29 - DNA, 26 bp, Artificial Sequence
  Primer
gcctttgtttggcaagctgctgtaag

SEQ ID NO: 30 - DNA, 25 bp, Artificial Sequence
  Primer
ggcattgctcgtattcacggtcttg

SEQ ID NO: 31 - DNA, 23 bp, Artificial Sequence
  Primer
ccactctggaaacggagataccc

SEQ ID NO: 32 - DNA, 24 bp, Artificial Sequence
  Primer
ctcgttttttatcagaagcttgtg

SEQ ID NO: 33 - DNA, 22 bp, Artificial Sequence
  Primer
gatggccctccatggattcacc

SEQ ID NO: 34 - DNA, 25 bp, Artificial Sequence
  Primer
gaggattaatgtcagatcctcaagg

SEQ ID NO: 35 - DNA, 23 bp, Artificial Sequence
  Primer
gatagacatcggtactccagtgc

SEQ ID NO: 36 - DNA, 23 bp, Artificial Sequence
  Primer
gttacacaacaaccccttagagg

SEQ ID NO: 37 - DNA, 23 bp, Artificial Sequence
  Primer
gcacaaattccgctttttatagg

SEQ ID NO: 38 - DNA, 26 bp, Artificial Sequence
  Primer
gcccaagcaagacttactatatccat

SEQ ID NO: 39 - DNA, 20 bp, Artificial Sequence
  Primer
ccagttcaaatccgggtgtc

SEQ ID NO: 40 - DNA, 19 bp, Artificial Sequence
  Primer
taccaagggctatagtcat

SEQ ID NO: 41 - DNA, 20 bp, Artificial Sequence
  Primer
gcccttttaactcagtggta

SEQ ID NO: 42 - DNA, 20 bp, Artificial Sequence
  Primer
aacccgcatcttctccttgg

SEQ ID NO: 43 - DNA, 20 bp, Artificial Sequence
  Primer
gagagagagggattcgaacc

SEQ ID NO: 44 - DNA, 20 bp, Artificial Sequence
  Primer
ttcgttcgccggaaccagaa

SEQ ID NO: 45 - DNA, 20 bp, Artificial Sequence
  Primer
aagaatgcccatgttgtggc

SEQ ID NO: 46 - DNA, 24 bp, Artificial Sequence
  Primer
cctattacagagatggtgcgattt

SEQ ID NO: 47 - DNA, 20 bp, Artificial Sequence
  Primer
cgatgcatatgtagaaagcc

SEQ ID NO: 48 - DNA, 23 bp, Artificial Sequence
  Primer
tttttatgaaatacaagatgctc

SEQ ID NO: 49 - DNA, 20 bp, Artificial Sequence
  Primer
cgaaatcggtagacgctacg

SEQ ID NO: 50 - DNA, 26 bp, Artificial Sequence
  Primer
gaaggaaggagacaaaaaattgaggc

SEQ ID NO: 51 - DNA, 20 bp, Artificial Sequence
  Primer
cgaaccgtagaccttctcgg

SEQ ID NO: 52 - DNA, 20 bp, Artificial Sequence
  Primer
gtaaaatcaagtccaccgcg

SEQ ID NO: 53 - DNA, 23 bp, Artificial Sequence
  Primer
tctaggatttacatatacaacat

SEQ ID NO: 54 - DNA, 22 bp, Artificial Sequence
  Primer
ctaataagaagcctaatgaacc

SEQ ID NO: 55 - DNA, 22 bp, Artificial Sequence
  Primer
gcaggtaaaagagtaattgaac

SEQ ID NO: 56 - DNA, 22 bp, Artificial Sequence
  Primer
tactcatttttgtacttgctgt

SEQ ID NO: 57 - DNA, 20 bp, Artificial Sequence
  Primer
gcatctgttattttggcaca

SEQ ID NO: 58 - DNA, 24 bp, Artificial Sequence
  Primer
accatagaaacgaaggaacccact

SEQ ID NO: 59 - DNA, 23 bp, Artificial Sequence
  Primer
gctcgtattttatctttgttacc

SEQ ID NO: 60 - DNA, 26 bp, Artificial Sequence
  Primer
ccccttggactgctacgaaaaacacc

SEQ ID NO: 61 - DNA, 22 bp, Artificial Sequence
  Primer
tgccttggtatcgtgttcatac

SEQ ID NO: 62 - DNA, 21 bp, Artificial Sequence
  Primer
cccagaaataccttgtttacg

SEQ ID NO: 63 - DNA, 20 bp, Artificial Sequence
  Primer
tggggaactactcctttgat

SEQ ID NO: 64 - DNA, 22 bp, Artificial Sequence
  Primer
cgaggtataatgacagaccgag

SEQ ID NO: 65 - DNA, 20 bp, Artificial Sequence
  Primer
attctacgtgcacccttacg

SEQ ID NO: 66 - DNA, 20 bp, Artificial Sequence
  Primer
gggcatctaccattataccc

SEQ ID NO: 67 - DNA, 24 bp, Artificial Sequence
  Primer
agtctgaaaccaagtggatttatt

SEQ ID NO: 68 - DNA, 26 bp, Artificial Sequence
  Primer
gaagatacaggagcgaaacaatcaac

SEQ ID NO: 69 - DNA, 23 bp, Artificial Sequence
  Primer
gcttatgacctccccctctatgc

SEQ ID NO: 70 - DNA, 26 bp, Artificial Sequence
  Primer
tcttctagagaatctcctaattgttc

SEQ ID NO: 71 - DNA, 26 bp, Artificial Sequence
  Primer
cttcgaatatggaattcaaagggatc

SEQ ID NO: 72 - DNA, 24 bp, Artificial Sequence
  Primer
ctcaaacaagcatgaaacgtatgc

SEQ ID NO: 73 - DNA, 22 bp, Artificial Sequence
  Primer
gagattttgagtctcgcgtgtc

SEQ ID NO: 74 - DNA, 26 bp, Artificial Sequence
  Primer
tcactgcttatatacccggtattggc

SEQ ID NO: 75 - DNA, 26 bp, Artificial Sequence
  Primer
gtgcaaaagctctatttgcctctgcc

SEQ ID NO: 76 - DNA, 26 bp, Artificial Sequence
  Primer
atcgaaagttggatctacattggatc

SEQ ID NO: 77 - DNA, 26 bp, Artificial Sequence
  Primer
cgacactgacactgagagacgaaagc

SEQ ID NO: 78 - DNA, 26 bp, Artificial Sequence
  Primer
cgctaccttaggaccgttatagttac

SEQ ID NO: 79 - DNA, 26 bp, Artificial Sequence
  Primer
gaaactaagtggaggtccgaaccgac

SEQ ID NO: 80 - DNA, 26 bp, Artificial Sequence
  Primer
agtggatccctcttgttcctgtttag

SEQ ID NO: 81 - DNA, 25 bp, Artificial Sequence
  Primer
acagccgaccgctctaccactgagc

SEQ ID NO: 82 - DNA, 25 bp, Artificial Sequence
  Primer
ggatcatacctttcattccacttcc

SEQ ID NO: 83 - DNA, 26 bp, Artificial Sequence
  Primer
atttcatctttggaccaaaaacaagc

SEQ ID NO: 84 - DNA, 23 bp, Artificial Sequence
  Primer
gctaaacaaattgcttctgctcc

SEQ ID NO: 85 - DNA, 26 bp, Artificial Sequence
  Primer
ggtcaatcttttaggaatagggttac

SEQ ID NO: 86 - DNA, 26 bp, Artificial Sequence
  Primer
ggacttcttatgtcgggatatggatc

SEQ ID NO: 87 - DNA, 26 bp, Artificial Sequence
  Primer
ctgcgcttccactatatcaactgtac

SEQ ID NO: 88 - DNA, 20 bp, Artificial Sequence
  Primer
tgaaaccttggcatatatct

SEQ ID NO: 89 - DNA, 22 bp, Artificial Sequence
  Primer
aatttcgaggttcttatttact

SEQ ID NO: 90 - DNA, 21 bp, Artificial Sequence
  Primer
gacgatactgtaggggaggtc

SEQ ID NO: 91 - DNA, 20 bp, Artificial Sequence
  Primer
ccatagaatacgaccctaat

SEQ ID NO: 92 - DNA, 20 bp, Artificial Sequence
  Primer
ctagcactgaaaaccgtctt

SEQ ID NO: 93 - DNA, 21 bp, Artificial Sequence
  Primer
aaagaagcacggtcaaatagg

SEQ ID NO: 94 - DNA, 20 bp, Artificial Sequence
  Primer
gcaacaacaaggtgtcatgg

SEQ ID NO: 95 - DNA, 21 bp, Artificial Sequence
  Primer
tgtgtactaccggcctactgc

SEQ ID NO: 96 - DNA, 20 bp, Artificial Sequence
  Primer
ttctgctaaaccgatcgtgg

SEQ ID NO: 97 - DNA, 21 bp, Artificial Sequence
  Primer
ggtcgatccacaatttaaacg

SEQ ID NO: 98 - DNA, 20 bp, Artificial Sequence
  Primer
gcacttgctcctttgtaccc

SEQ ID NO: 99 - DNA, 20 bp, Artificial Sequence
  Primer
gaacctaacctcgctccaca

SEQ ID NO: 100 - DNA, 20 bp, Artificial Sequence
  Primer
aaatggtagctgcgaggaga

SEQ ID NO: 101 - DNA, 20 bp, Artificial Sequence
  Primer
gtctgtaccttcgccaaagc

SEQ ID NO: 102 - DNA, 20 bp, Artificial Sequence
  Primer
tcctcagagaactccagcgt

SEQ ID NO: 103 - DNA, 20 bp, Artificial Sequence
  Primer
cttcttcctaagccgagggt

SEQ ID NO: 104 - DNA, 21 bp, Artificial Sequence
  Primer
ttgatgatagaacgcaactcg

SEQ ID NO: 105 - DNA, 20 bp, Artificial Sequence
  Primer
agttgcaggattgttcgctt

SEQ ID NO: 106 - DNA, 20 bp, Artificial Sequence
  Primer
cgactgcaagagttggcaat

SEQ ID NO: 107 - DNA, 23 bp, Artificial Sequence
  Primer
tgatacagaatatggcgaacttt

SEQ ID NO: 108 - DNA, 20 bp, Artificial Sequence
  Primer
ccgcttcatcattgaggttt

SEQ ID NO: 109 - DNA, 20 bp, Artificial Sequence
  Primer
aagatggcatatgggattgg

SEQ ID NO: 110 - DNA, 20 bp, Artificial Sequence
  Primer
tgaatcggaggaagtgaatg

SEQ ID NO: 111 - DNA, 20 bp, Artificial Sequence
  Primer
cttctctctccaccgcagac

SEQ ID NO: 112 - DNA, 20 bp, Artificial Sequence
  Primer
acagttggatatggtggcgt

SEQ ID NO: 113 - DNA, 20 bp, Artificial Sequence
  Primer
cgttgcttagtctcgcactg

SEQ ID NO: 114 - DNA, 22 bp, Artificial Sequence
  Primer
ggttgatccgacactattacga

SEQ ID NO: 115 - DNA, 22 bp, Artificial Sequence
  Primer
ttgttgctctctcgagttcttt

SEQ ID NO: 116 - DNA, 18 bp, Artificial Sequence
  Primer
gcagtcgactcattggca

SEQ ID NO: 117 - DNA, 24 bp, Artificial Sequence
  Primer
gacaacaatcagtaaaggaaacga

SEQ ID NO: 118 - DNA, 20 bp, Artificial Sequence
  Primer
aatgcaagaccctgtcaacc

SEQ ID NO: 119 - DNA, 20 bp, Artificial Sequence
  Primer
aacaaaccgctttccattct

SEQ ID NO: 120 - DNA, 20 bp, Artificial Sequence
  Primer
gaattaggcgctttgggaat

SEQ ID NO: 121 - DNA, 20 bp, Artificial Sequence
  Primer
ttaggcggcggtattcttat

SEQ ID NO: 122 - DNA, 20 bp, Artificial Sequence
  Primer
tatgcctcaatcccttacgc

SEQ ID NO: 123 - DNA, 20 bp, Artificial Sequence
  Primer
aagctgccctagctcaatca

SEQ ID NO: 124 - DNA, 23 bp, Artificial Sequence
  Primer
aacatcaccatttccacaagttt

SEQ ID NO: 125 - DNA, 20 bp, Artificial Sequence
  Primer
catttagccgggaacattca

SEQ ID NO: 126 - DNA, 20 bp, Artificial Sequence
  Primer
catgggatacacacgcaaag

SEQ ID NO: 127 - DNA, 27 bp, Artificial Sequence
  Primer
tgacagctaagctaataacagtaaatg

SEQ ID NO: 128 - DNA, 21 bp, Artificial Sequence
  Primer
ggactttggagtgtcaaatgc

SEQ ID NO: 129 - DNA, 20 bp, Artificial Sequence
  Primer
agccagccaccaaatttatc

SEQ ID NO: 130 - DNA, 20 bp, Artificial Sequence
  Primer
ggaacattgctcaagcccta

SEQ ID NO: 131 - DNA, 24 bp, Artificial Sequence
  Primer
tttctttctgtctgatgcttcaat

SEQ ID NO: 132 - DNA, 20 bp, Artificial Sequence
  Primer
ttgtccatctcacttgctgc

SEQ ID NO: 133 - DNA, 18 bp, Artificial Sequence
  Primer
acgctagagcatccaaca

SEQ ID NO: 134 - DNA, 18 bp, Artificial Sequence
  Primer
tagtgaaaggcaagcagg

SEQ ID NO: 135 - DNA, 20 bp, Artificial Sequence
  Primer
tcaaatgagggttgtagcca

SEQ ID NO: 136 - DNA, 21 bp, Artificial Sequence
  Primer
tgcaatggctacacaagaaga

SEQ ID NO: 137 - DNA, 20 bp, Artificial Sequence
  Primer
ttgaacaccaattgcggtaa

SEQ ID NO: 138 - DNA, 20 bp, Artificial Sequence
  Primer
aaattcttgggtcatggtgg

SEQ ID NO: 139 - DNA, 20 bp, Artificial Sequence
  Primer
aggaggcgaagaaagaggag

SEQ ID NO: 140 - DNA, 20 bp, Artificial Sequence
  Primer
cccatgaattcgtaacagca

SEQ ID NO: 141 - DNA, 20 bp, Artificial Sequence
  Primer
aatgtctgcccaatcgaaag

SEQ ID NO: 142 - DNA, 20 bp, Artificial Sequence
  Primer
cgaataacgacactcgaacg

SEQ ID NO: 143 - DNA, 20 bp, Artificial Sequence
  Mitochondrial DNA sequence
aaaaccaatatcataacttt

SEQ ID NO: 144 - DNA, 20 bp, Artificial Sequence
  Mitochondrial DNA sequence
aaaaccaatctcataacttt

SEQ ID NO: 145 - DNA, 20 bp, Artificial Sequence
  Mitochondrial DNA sequence
gaaacgaagtggagcgaggg

SEQ ID NO: 146 - DNA, 20 bp, Artificial Sequence
  Mitochondrial DNA sequence
gaaacgaagcggagcgaggg

SEQ ID NO: 147 - DNA, 20 bp, Artificial Sequence
  Mitochondrial DNA sequence
aaaaaatttatctcttttta

SEQ ID NO: 148 - DNA, 20 bp, Artificial Sequence
  Mitochondrial DNA sequence
aaaaaatttctctcttttta

SEQ ID NO: 149 - DNA, 20 bp, Artificial Sequence
  Mitochondrial DNA sequence
ctttgttctgtagtctttca

SEQ ID NO: 150 - DNA, 20 bp, Artificial Sequence
  Mitochondrial DNA sequence
ctttgttctctagtctttca

SEQ ID NO: 151 - DNA, 20 bp, Artificial Sequence
  Mitochondrial DNA sequence
ctcccatttgaaagattatt

SEQ ID NO: 152 - DNA, 20 bp, Artificial Sequence
  Mitochondrial DNA sequence
ctcccattttaaagattatt

SEQ ID NO: 153 - DNA, 20 bp, Artificial Sequence
  Mitochondrial DNA sequence
tgcacctccgtacaagtgct

SEQ ID NO: 154 - DNA, 10 bp, Artificial Sequence
  Mitochondrial DNA sequence
tgcacgtgct

SEQ ID NO: 155 - DNA, 20 bp, Artificial Sequence
  Mitochondrial DNA sequence
ttccggctgtttccgtcatt

SEQ ID NO: 156 - DNA, 10 bp, Artificial Sequence
  Mitochondrial DNA sequence
ttccgtcatt

SEQ ID NO: 157 - DNA, 20 bp, Artificial Sequence
  Mitochondrial DNA sequence
aagaaaaaagaaaagtcgag

SEQ ID NO: 158 - DNA, 20 bp, Artificial Sequence
  Mitochondrial DNA sequence
aagaaaaaatcaaagtcgag

SEQ ID NO: 159 - DNA, 20 bp, Artificial Sequence
  Mitochondrial DNA sequence
ggatcagactactcctggtg

SEQ ID NO: 160 - DNA, 20 bp, Artificial Sequence
  Mitochondrial DNA sequence
ggatcagaccactcctggtg

SEQ ID NO: 161 - DNA, 20 bp, Artificial Sequence
  Mitochondrial DNA sequence
agcaaaactcgaacggatag

SEQ ID NO: 162 - DNA, 20 bp, Artificial Sequence
  Mitochondrial DNA sequence
agcaaaactagaacggatag

SEQ ID NO: 163 - DNA, 23 bp, Artificial Sequence
  Plastid sequence polymorphism
tagagaatcaaaattgatttacc

SEQ ID NO: 164 - DNA, 23 bp, Artificial Sequence
  Plastid sequence polymorphism
tagaaaatcaaaatcgatttacc

SEQ ID NO: 165 - DNA, 23 bp, Artificial Sequence
  Plastid sequence polymorphism
tctgttatggatttcgttgctaa

SEQ ID NO: 166 - DNA, 23 bp, Artificial Sequence
  Plastid sequence polymorphism
tatgttatggatttcgttgttca

SEQ ID NO: 167 - DNA, 20 bp, Artificial Sequence
  Plastid sequence polymorphism
tcaaaatcaaataatctgta

SEQ ID NO: 168 - DNA, 14 bp, Artificial Sequence
  Plastid sequence polymorphism
tcaaataatccgta

SEQ ID NO: 169 - DNA, 23 bp, Artificial Sequence
  Plastid sequence polymorphism
caacgcctacattgcatttgcag

SEQ ID NO: 170 - DNA, 23 bp, Artificial Sequence
  Plastid sequence polymorphism
caatgcctatattgcatttgcag

SEQ ID NO: 171 - DNA, 23 bp, Artificial Sequence
  Plastid sequence polymorphism
ttagaaatgtttcccttgattga

SEQ ID NO: 172 - DNA, 23 bp, Artificial Sequence
  Plastid sequence polymorphism
ttataaatgttccccttgattga

SEQ ID NO: 173 - DNA, 20 bp, Artificial Sequence
  Plastid sequence polymorphism
agtaaattcagccaactaag

SEQ ID NO: 174 - DNA, 20 bp, Artificial Sequence
  Plastid sequence polymorphism
agtcaattcagccaacaaag

SEQ ID NO: 175 - DNA, 18 bp, Artificial Sequence
  Plastid sequence polymorphism
atctcgactcctttcaat

SEQ ID NO: 176 - DNA, 23 bp, Artificial Sequence
  Plastid sequence polymorphism
atcgcgactcctttcctttcaat

(19) United States
(12) Patent Application Publication
(10) Pub. No.: US 2014/0075592 A1
(43) Pub. Date: Mar. 13, 2014

(54) Intercellular Transfer of Organelles in Plants for Horizontal Transfer of DNA Expressing Proteins of Interest

(71) Applicant: RUTGERS, THE STATE UNIVERSITY OF NEW JERSEY, NEW BRUNSWICK, NJ (US)

(72) Inventors: Pal Maliga, East Brunswick, NJ (US); Zora S. Maliga, East Brunswick, NJ (US); Gregory N. Thyssen, New Brunswick, NJ (US)

(21) Appl. No.: 13/930,378

(22) Filed: Jun. 28, 2013

Related U.S. Application Data
(63) Continuation-in-part of application No. PCT/US11/68153, filed on Dec. 30, 2011.
(60) Provisional application No. 61/428,672, filed on Dec. 30, 2010.

(51) Int. Cl. C12N 15/82

(52) U.S. Cl. CPC C12N 15/8214
USPC 800/278; 800/298

(57) ABSTRACT

Compositions and methods for effecting horizontal gene transfer in plants are disclosed.

This application is a §365 Application of PCT/US11/68153 filed Dec. 30, 2011, which in turn claims priority to U.S. Provisional Application 61/428,672 filed Dec. 30, 2010, the entire disclosures of each being incorporated herein by reference as though set forth in full.

### FIELD OF THE INVENTION

The present invention relates to plant genetic engineering and particularly to methods for horizontal gene transfer in higher plants.

### BACKGROUND OF THE INVENTION

Several publications and patent documents are cited throughout the specification in order to describe the state of the art to which this invention pertains. Each of these citations is incorporated herein by reference as though set forth in full.

Cells within a multicellular organism are connected by cytoplasmic bridges, which are termed plasmodesmata in plants (1) and tunneling nanotubes in animals (2). Plasmodesmata were shown to actively and passively regulate intercellular trafficking of viral proteins, transcription factors, phloem proteins, mRNA and sRNA in plants (1, 3). An important, recent development was the demonstration of the exchange of genetic material between cells in plant tissue grafts (4). However, there is no report yet on the intercellular movement of DNA-containing organelles, plastids and mitochondria, between plant cells.

During the past few years, supracellularity has emerged as a trait common to all life. Once thought to be a feature unique to plants, the physical continuity of cytoplasm and plasma membranes between neighboring cells has been observed in animal cells (2). These tunneling nanotubes were shown to be the conduits of active transport of organelles and cytoplasmic molecules between cells. Particularly relevant for this work, is the direct observation of transport of mitochondria through tunneling nanotubes in animal cells (5, 6). Tunneling nanotubes, filopodia-like cytoplasmic bridges have also been observed linking unrelated bacterial cells and therefore may represent a universal mechanism for cellular communication and interdependence (7).

Clearly, modulation of this process would represent an advance in the art in the creation of transplastomic plants.

### SUMMARY OF THE INVENTION

In accordance with the present invention, a method for effecting intercellular transfer of organelles in plants for the creation of transgenic plants exhibiting desirable characteristics is provided. An exemplary method entails joining a root stock of a first plant and a scion from a second plant, said first and second plants comprising distinct plastid and nuclear genetic markers; culturing for a suitable period for grafting to occur; fragmenting or slicing the graft region and transferring said fragment or slices to a plant regeneration medium and selecting for cells expressing the nuclear and plastid genetic markers from said first and second plants. In one embodiment, the method entails decapitating the rootstock of a first plant, splitting the stem of said root stock and inserting a wedge shaped stem of scion from a second plant in the opening in the root stock, said first and second plants comprising distinct plastid and nuclear genetic markers; and culturing the graft plant for a suitable period for grafting to occur; then following the protocol above. The method can also comprise characterization of the size and type of DNA transferred. In a preferred embodiment, the organelle is a plastid and the method results in complete transfer of the plastid genome. In a particularly preferred aspect, the transferred plastid genome comprises at least one heterologous or endogenous DNA molecule expressing a protein of interest, e.g., a protein conferring herbicide or drought resistance. Other proteins of interest include without limitation, a fluorescent protein, an antibody, a cytokine, an interferon, a hormone, a selectable marker protein, a coagulation factor and/or an enzyme. Also provided are transgenic plants generated using the foregoing methods.

### BRIEF DESCRIPTION OF THE DRAWINGS

FIG. 1. Phenotypes of the graft partners and the G1 graft transfer plant. (A) Partner P1 is N. tabacum (2N=48) with a nuclear gentamycin resistance transgene and wild-type N. tabacum plastids and mitochondria. Partner P2 has a wild-type N. sylvestris (2N=24) nuclear genome, N. undulata plastids with aadA transgenes for spectinomycin selection and the aurea young leaf color phenotype (bar^{au} gene) and N. undulata mitochondria that confer cytoplasmic male sterility (CMS-92). Shown is also the G1 plant and its markers. Black bar=10 cm (B) Flower morphology of the P1 and P2 partners and G1 PGT plant. White bar=1 cm

FIG. 2. Identification of plastid graft transfer events. (A) Grafted plant. Note that the P2 scion shown here is green because the expression of the bar^{au} gene is restricted to fast growing tissue and is sensitive to environmental conditions. (B) Selection in cultures of one- to two-mm graft sections for gentamycin- and spectinomycin-resistance. On the left are stem sections from above (P2) and below (P1) the graft and on the right from the graft region. Note a green, proliferating callus that yielded the G4 PGT plants.

FIG. 3. SSR markers confirm N. tabacum chromosomes in the G4 plant by testing each of the 24 chromosomes (numbered 1-24). Lanes are marked with s, G and t for the P2, G1 and P1 plants (see caption to FIG. 1). Some markers do not amplify the N. sylvestris template (8). White dots mark the 200-bp fragment of the 20-bp molecular weight ladder.

FIG. 4. Identification of the source of mtDNA in the PGT plants. (A) Schematic representation of the tobacco mtDNA master circle with the position of polymorphic regions marked. Repeated regions are marked with boxes. (B) Mitochondrial DNA sequence polymorphisms. (C) Map position of polymorphic sites relative to the sequencing primers and gene features.

FIG. 5. Identification of the N. undulata plastids in the PGT plants. (A) Identity plots of the plastid genomes of the transplastomic P2 partner carrying N. undulata ptDNA (u) with the aadA and bar^{au} transgenes (JN563930), the G1, G3, G4 (G) PGT plants and the P1 partner with N. tabacum ptDNA (t; Z00044) aligned with the mVISTA program using a 500-bp sliding window. Above the map are shown the positions of the DNA probes (#1 through #6) and DNA polymorphisms (*1 through *7). (B) Plastid DNA sequence polymorphisms. For map position see FIG. 5A. (C) DNA gel blot to identify RFLP markers in ptDNA. For probes see FIG. 5A.

FIG. 6. Model for cell-to-cell movement of plastids via initial cytoplasmic connection in graft junctions. (A) Cells at graft junction reconnect by plasmodesmata. Arrows point to sites where opposite parts of the contact walls are synchronously thinned (9). These are future sites of plasmodesmata. Proplastids (ovals), mitochondria (small circles) and nuclei (large circles) are identified in scion and rootstock. Ns, N. sylvestris; Nt, N. tabacum, Nu. N. undulata; CMS, cytoplasmic male sterile. (B) Proplastid is transferred via initial cytoplasmic connection. (C) Transferred spectinomycin resistant plastid takes over on selective medium. Note that the cells derive from the bottom cell in FIG. 6B.

### DETAILED DESCRIPTION OF THE INVENTION

Land plants developed highly sophisticated intercellular channels or plasmodesmata, which mediate cell-to-cell movement of nutrients, hormones and information macromolecules. Functional equivalents of plasmodesmata in animal cells are the tunneling nanotubes, which were shown to mediate intercellular trafficking of organelles. Our objective was to test whether or not organelles move between cells in plants. As our experimental approach, we grafted two different species of tobacco, Nicotiana tabacum and Nicotiana sylvestris. Grafting triggers formation of new plasmodesmatal connections between the genetically distinct plants creating an opportunity for cell-to-cell organelle movement. We selected in tissue culture of the graft junctions for clonal lines, in which gentamycin resistance encoded in the N. tabacum nucleus was combined with spectinomycin resistance encoded in N. sylvestris plastids.

Here we report cell-to-cell movement of the entire 161-kb plastid genome in these plants, most likely in the form of intact plastids. We also report that the related mitochondria were absent, suggesting independent movement of the two DNA-containing organelles. Acquisition of plastids from neighboring cells via plasmodesmata provides a novel mechanism by which cells may be re-populated with functioning organelles. Our finding confirms the universal nature of intercellular organelle trafficking thus far documented only in mammalian cells and enables new biotechnological applications. Plastid transformation currently is a tissue culture dependent protocol that can be performed only with tissue-culture responsive genetic lines. Introduction of transformed plastid genomes into commercially useful lines requires repeated cycles of backcrosses. Inter-cellular transfer of organellar DNA in tissue grafts enables one-step transfer of plastid genomes in the absence of the transfer of nuclear genetic information, eliminating the need for backcrosses. Furthermore, graft transfer of plastidsis possible between sterile plants lacking flowers and between sexually incompatible genetic lines.

Inter-cellular transfer of plastid DNA is an example of horizontal gene transfer, defined as any process in which the recipient organism acquires genetic material from a donor organism by asexual means. Well studied is the role of massive horizontal gene transfer in the evolution of mitochondria and plastids from an alpha-proteobacterium and a cyanobacterium, respectively (Abdallah et al., 2000; Keeling and Palmer, 2008; Gross and Bhattacharya, 2009). Additional examples of horizontal gene transfer during evolution include prokaryote-eukaryote transfers, eukaryote-eukaryote transfers, eukaryote-prokaryote transfers and horizontal gene transfer in the plant cytoplasm (Richardson and Palmer, 2007; Keeling and Palmer, 2008; Bock, 2009).

The following definitions are provided to facilitate an understanding of the invention.

“Heteroplastomic” refers to the presence of a mixed population of different plastid genomes within a single plastid or in a population of plastids contained in plant cells or tissues.

“Homoplastomic” refers to a pure population of plastid genomes, either within a plastid or within a population contained in plant cells and tissues. Homoplastomic plastids, cells or tissues are genetically stable because they contain only one type of plastid genome. Hence, they remain homoplastomic even after the selection pressure has been removed, and selfed progeny are also homoplastomic. For purposes of the present invention, heteroplastomic populations of genomes that are functionally homoplastomic (i.e., contain only minor populations of wild-type DNA or transformed genomes with sequence variations) may be referred to herein as “functionally homoplastomic” or “substantially homoplastomic.” These types of cells or tissues can be readily purified to a homoplastomic state by continued selection.

“Plastome” refers to the genome of a plastid.

“Transplastome” refers to a transformed plastid genome.

“Transformation of plastids” refers to the stable integration of transforming DNA into the plastid genome that is transmitted to the seed progeny of plants containing the transformed plastids.

A “selectable marker gene” refers to a gene that upon expression confers a phenotype by which successfully transformed plastids or cells or tissues carrying the transformed plastid can be identified. Selectable marker genes as used herein can confer resistance to a selection agent in tissue culture and/or confer a phenotype which is identifiable upon visual inspection. Thus, in one embodiment the selectable marker gene can act as both the selection agent and the agent which enables visual identification of cells comprising transformed plastids. In an alternative embodiment, the selectable marker encoding nucleic acid comprises two sequences, one encoding a molecule that renders cells resistant to a selection agent in tissue culture and another that enables visual identification of cells comprising transformed plastids.

“Transforming DNA” refers to homologous DNA, or heterologous DNA flanked by homologous DNA, which when introduced into plastids becomes part of the plastid genome by homologous recombination.

“Agroinfiltration” refers to Agrobacterium mediated T-DNA transfer. Specifically, this process involves vacuum treatment of leaf segments in an Agrobacterium suspension and a subsequent release of vacuum, which facilitates entry of bacterium cells into the inter-cellular space.

“T-DNA” refers to the transferred-region of the Ti (tumor-inducing) plasmid of Agrobacterium tumefaciens. Ti plasmids are natural gene transfer systems for the introduction of heterologous nucleic acids into the nucleus of higher plants.

A “plant sector” refers to a region or a full leaf of a plant that is visually identifiable due to expression of a selectable marker gene or the excision of a selectable marker gene in accordance with the present invention.

“Operably linked” refers to two different regions or two separate genes spliced together in a construct such that both regions will function to promote gene expression and/or protein translation.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

A “replicon” is any genetic element, for example, a plasmid, cosmid; bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis.

All amino-acid residue sequences represented herein conform to the conventional left-to-right amino-terminus to carboxy-terminus orientation.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion, biolistic bombardment and the like.

A “clone” or “clonal cell population” is a population of cells derived from a single cell or common ancestor by mitosis.

A “cell line” is a clone of a primary cell or cell population that is capable of stable growth in vitro for many generations.

The following materials and methods are provided to facilitate the practice of the present invention.

### Materials and Methods

Partner P1 (Nt-pHC19) has an allotetraploid Nicotiana tabacum cv. Petit Havana (2N=48) nucleus with the aacC1 transgene for gentamycin resistance and wild-type N. tabacum plastid and mitochondrial genomes (10). Partner P2 (Ns-pCK2-6W2) has a wild-type diploid N. sylvestris TW137 (2N=24) nuclear genome, N. undulata plastids with aadA transgenes for spectinomycin selection and the aurea young leaf color phenotype (bar^{au} gene), and cytoplasmic male sterile (CMS-92) mitochondria from N. undulata (11). For grafting, the plants were grown aseptically on a medium containing MS salts and 3% sucrose (12). Plants were regenerated from the graft junctions on RMOP shoot regeneration media supplemented with 500 mg/L spectinomycin and 100 mg/L gentamycin (12). Southern probing for ptDNA polymorphisms was carried out using six previously identified polymorphic regions (13). Organellar DNA was amplified using total cellular DNA as a template (14) using appropriate PCR primers (Table S1, Table S2). Primer design for ptDNA was based on GenBank Accession Z00044 and JN563929 and for mtDNA on GenBank Accession BA000042. The plastid genomes were amplified in 34 PCR reactions using primers listed in Table S3. DNA sequence was determined on an Illumina Genome Analyzer II using 80 bp paired-end (500 bp insert) library. Total leaf DNA fragments of P1, P2, G1, G3 and G4 plants were also analyzed on a SOLiD 5500×1 sequencer using 76-nucleotide reads. Reference guided assembly was essentially carried out as described (15). Nuclear SSR markers (8) were amplified using primers listed in Table S4.

TABLE S1
Plastid primers for testing ptDNA polymorphic sites between
N. tabacum and N. undulata

| Pair | Primer | Position | Strand | Gene | Sequence |
|---|---|---|---|---|---|
| *1 | 12upF | 12907 | F | atfP | TCTTACTTAGAATAGGTCGTCGATTCAGCA |
| *1 | 14upR | 14098 | R | atfP | CCACTGATTTCTGCCGCTTCCGTT |
| *2 | 27upF | 27875 | F | rpoB-trnC | ACACATTCCAACCTGCTTGAATACCA |
| *2 | 29upR | 29210 | R | rpoB-trnC | TCTTCCGCCCCCTTCCACAACTAT |
| *3 | 48upF | 48971 | F | trnL | GAGACATTCCTCCGCTTTCAGGCG |
| *3 | 49upR | 49945 | R | trnL | TGGAACCGCTAAGGAAAGGGGGTC |
| *4 | 60upF | 60806 | F | accD | AACGGCATTCCCGTAGCAATTGGG |
| *4 | 62upR | 62222 | R | accD | GGATGAGATTGGGTCCCAGCGGAT |
| *5 | 83upF | 83888 | F | ndhF | TTTCCACCACGACGTGCATTTCGT |
| *5 | 85upR | 85414 | R | ndhF | TACAAATTGCGGGGCGTATCGACG |
| *6 | 111upF | 111916 | F | ndhE-ndhG | TCGGAAGAAAGGTGGGATCCGGAC |
| *6 | 113upR | 113293 | R | ndhE-ndhG | TGGTATGGGGTCTTATCGAAGCGC |

TABLE S2
Mitochondrial primers for testing mtDNA polymorphic sites
between N. tabacum and N. undulata

| Pair | Primer | Position | Strand | Gene | Sequence |
|---|---|---|---|---|---|
| 1 | mt-0-F | 690 | F | orf125a | CCCCGCCCAGTAGTGCCTCT |
| 1 | mt-4-R | 4334 | R | orf125a | CCGCGGGCATCGCGATAAGT |
| 2 | mt-100-F | 100070 | F | orf129b | CGGCCATCCTGGTCCTCAGGA |
| 2 | mt-104-R | 104811 | R | orf129b | TGGGGACTCGCACGAGGAGG |
| 3 | mt-180-F | 180316 | F | nad4 | GGCAGGAGCGCAACGACCTT |
| 3 | mt-183-R | 183813 | R | nad4 | AGTCGGGTTGCTCACGCAGC |
| 4 | mt-201-F | 201586 | F | nad2 | TGGTGTGCTTCCTGCTCGCG |
| 4 | mt-204-R | 204759 | R | nad2 | TTTCTCCGTGCCCGTTCCGC |
| 5 | mt-222-F | 222140 | F | nad5 | AGGTGCCCGTAGTAGGCCGG |
| 5 | mt-226-R | 226463 | R | nad5 | TTGGGCTTGGCTCTGCTCGC |
| 6 | mt-306-F | 306203 | F | orf115-ccmFc | CACGACTCCCCCTCTCCCCG |
| 6 | mt-309-R | 309623 | R | orf115-ccmFc | TGCCCGATTCCCCGACCCAT |

TABLE S3
Plastid primers for PCR amplification of the Nicotiana
tabacum and N. undulata plastid genomes

| Pair | Primer | Position | Strand | Gene | Sequence |
|---|---|---|---|---|---|
| 1 | 0F | 14 | F | trnH | ACGGGAATTGAACCCGCGCA |
| 1 | 4R | 4410 | R | trnK | CGGGTTGCTAACTCAACGG |
| 2 | 3F | 3704 | F | trnK | TCAAATGATACATAGTGCGATACA |
| 2 | 8R | 8653 | R | trnS | CGAATCCCTCTCTTTCCG |
| 3 | 7F | 7989 | F | psbK | GCCTTTGTTTGGCAAGCTGCTGTAAG |
| 3 | 12R | 12042 | R | atpA | GGCATTGCTCGTATTCACGGTCTTG |
| 4 | 11F | 11052 | F | atpA | CCACTCTGGAAACGGAGATACCC |
| 4 | 16R | 16791 | R | rps2 | CTCGTTTTTTATCAGAAGCTTGTG |
| 5 | 15F | 15267 | F | atpI | GATGGCCCTCCATGGATTCACC |
| 5 | 20R | 20888 | R | rpoC2 | GAGGATTAATGTCAGATCCTCAAGG |
| 6 | 19F | 19971 | F | rpoC2 | GATAGACATCGGTACTCCAGTGC |
| 6 | 24R | 24612 | R | rpoB | GTTACACAACAACCCCTTAGAGG |
| 7 | 24F | 24069 | F | rpoC1 | GCACAAATTCCGCTTTTTATAGG |
| 7 | 29R | 29568 | R | ycf6 | GCCCAAGCAAGACTTACTATATCCAT |
| 8 | 28F | 28849 | F | trnC | CCAGTTCAAATCCGGGTGTC |
| 8 | 34R | 34493 | R | psbD | TACCAAGGGCTATAGTCAT |
| 9 | 33F | 33186 | F | trnT | GCCCTTTTAACTCAGTGGTA |
| 9 | 38R | 38115 | R | trnG | AACCCGCATCTTCTCCTTGG |
| 10 | 37F | 37147 | F | trnS | GAGAGAGAGGGATTCGAACC |
| 10 | 43R | 43484 | R | psaA | TTCGTTCGCCGGAACCAGAA |
| 11 | 41F | 41267 | F | psaA | AAGAATGCCCATGTTGTGGC |
| 11 | 46R | 46162 | R | ycf3 | CCTATTACAGAGATGGTGCGATTT |
| 12 | 45F | 45083 | F | ycf3 | CGATGCATATGTAGAAAGCC |
| 12 | 51R | 51022 | R | ndhJ | TTTTTATGAAATACAAGATGCTC |
| 13 | 49F | 49312 | F | trnL | CGAAATCGGTAGACGCTACG |
| 13 | 54R | 54971 | R | atpE | GAAGGAAGGAGACAAAAAATTGAGGC |
| 14 | 53F | 53776 | F | trnV | CGAACCGTAGACCTTCTCGG |
| 14 | 58R | 58198 | R | rbcL | GTAAAATCAAGTCCACCGCG |
| 15 | 57F | 57272 | F | atpB | TCTAGGATTTACATATACAACAT |
| 15 | 62R | 62754 | R | ycf4 | CTAATAAGAAGCCTAATGAACC |
| 16 | 61F | 61145 | F | accD | GCAGGTAAAAGAGTAATTGAAC |
| 16 | 66R | 66664 | R | psbL | TACTCATTTTTGTACTTGCTGT |
| 17 | 65F | 65219 | F | petA | GCATCTGTTATTTTGGCACA |
| 17 | 71R | 71704 | R | clpP | ACCATAGAAACGAAGGAACCCACT |
| 18 | 70F | 70727 | F | rps18 | GCTCGTATTTTATCTTTGTTACC |
| 18 | 76R | 76301 | R | psbB | CCCCTTGGACTGCTACGAAAAACACC |
| 19 | 74F | 74963 | F | psbB | TGCCTTGGTATCGTGTTCATAC |
| 19 | 78R | 78846 | R | petB | CCCAGAAATACCTTGTTTACG |
| 20 | 77F | 77212 | F | psbH | TGGGGAACTACTCCTTTGAT |
| 20 | 82R | 82676 | R | rps8 | CGAGGTATAATGACAGACCGAG |
| 21 | 81F | 81880 | F | rp136 | ATTCTACGTGCACCCTTACG |
| 21 | 86R | 86576 | R | rps19 | GGGCATCTACCATTATACCC |
| 22 | 85F | 85864 | F | rps3 | AGTCTGAAACCAAGTGGATTTATT |
| 22 | 89R | 89311 | R | YCF2 | GAAGATACAGGAGCGAAACAATCAAC |
| 23 | 88F | 88062 | F | rp12 | GCTTATGACCTCCCCCTCTATGC |
| 23 | 93R | 93140 | R | YCF2 | TCTTCTAGAGAATCTCCTAATTGTTC |
| 24 | 91F | 91131 | F | YCF2 | CTTCGAATATGGAATTCAAAGGGATC |
| 24 | 97R | 97636 | R | ndhB | CTCAAACAAGCATGAAACGTATGC |
| 25 | 96F | 96469 | f | trnL | GAGATTTTGAGTCTCGCGTGTC |
| 25 | 100R | 100782 | R | rps12 | TCACTGCTTATATACCCGGTATTGGC |
| 26 | 99F | 99552 | F | rps7 | GTGCAAAAGCTCTATTTGCCTCTGCC |
| 26 | 104R | 104797 | R | oriA | ATCGAAAGTTGGATCTACATTGGATC |
| 27 | 103F | 103454 | F | rrn16 | CGACACTGACACTGAGAGACGAAAGC |
| 27 | 108R | 108280 | R | rrn23 | CGCTACCTTAGGACCGTTATAGTTAC |
| 28 | 107F | 107056 | F | rrn23 | GAAACTAAGTGGAGGTCCGAACCGAC |
| 28 | 111R | 111882 | R | ORF350 | AGTGGATCCCTCTTGTTCCTGTTTAG |
| 29 | 110F | 110672 | F | trnN | ACAGCCGACCGCTCTACCACTGAGC |
| 29 | 114R | 114269 | R | ndhF | GGATCATACCTTTCATTCCACTTCC |
| 30 | 113F | 113036 | F | ndhF | ATTTCATCTTTGGACCAAAAACAAGC |
| 30 | 119R | 119286 | R | psaC | GCTAAACAAATTGCTTCTGCTCC |
| 31 | 117F | 117227 | F | ycf5 | GGTCAATCTTTTAGGAATAGGGTTAC |
| 31 | 123R | 123506 | R | ndhA | GGACTTCTTATGTCGGGATATGGATC |
| 32 | 122F | 122194 | F | ndhA | CTGCGCTTCCACTATATCAACTGTAC |
| 32 | 128R | 128835 | R | ycf1 | TGAAACCTTGGCATATATCT |
| 33 | 127F | 127391 | F | ycf1 | AATTTCGAGGTTCTTATTTACT |
| 33 | 132R | 132957 | R | trnR | GACGATACTGTAGGGGAGGTC |
| 34 | 154F | 154629 | F | rp12 | CCATAGAATACGACCCTAAT |
| 34 | 1R | 1533 | R | psbA | CTAGCACTGAAAACCGTCTT |

TABLE S4
Nuclear SSR Primers

| Chromosome | Primer | Strand | Sequence |
|---|---|---|---|
| 1 | PT30307 | F | AAAGAAGCACGGTCAAATAGG |
| 1 | PT30307 | R | GCAACAACAAGGTGTCATGG |
| 2 | PT30242 | F | TGTGTACTACCGGCCTACTGC |
| 2 | PT30242 | R | TTCTGCTAAACCGATCGTGG |
| 3b | PT30205 | F | GGTCGATCCACAATTTAAACG |
| 3b | PT30205 | R | GCACTTGCTCCTTTGTACCC |
| 4 | PT30272 | F | GAACCTAACCTCGCTCCACA |
| 4 | PT30272 | R | AAATGGTAGCTGCGAGGAGA |
| 5 | PT30471 | F | GTCTGTACCTTCGCCAAAGC |
| 5 | PT30471 | R | TCCTCAGAGAACTCCAGCGT |
| 6 | PT30087 | F | CTTCTTCCTAAGCCGAGGGT |
| 6 | PT30087 | R | TTGATGATAGAACGCAACTCG |
| 7 | PT30138 | F | AGTTGCAGGATTGTTCGCTT |
| 7 | PT30138 | R | CGACTGCAAGAGTTGGCAAT |
| 8a | PT30167 | F | TGATACAGAATATGGCGAACTTT |
| 8a | PT30167 | R | CCGCTTCATCATTGAGGTTT |
| 9 | PT30140 | F | AAGATGGCATATGGGATTGG |
| 9 | PT30140 | R | TGAATCGGAGGAAGTGAATG |
| 10 | PT30482 | F | CTTCTCTCTCCACCGCAGAC |
| 10 | PT30482 | R | ACAGTTGGATATGGTGGCGT |
| 11 | PT30008 | F | CGTTGCTTAGTCTCGCACTG |
| 11 | PT30008 | R | GGTTGATCCGACACTATTACGA |
| 12 | PT30098 | F | TTGTTGCTCTCTCGAGTTCTTT |
| 12 | PT30098 | R | GCAGTCGACTCATTGGCA |
| 13 | PT30342 | F | GACAACAATCAGTAAAGGAAACGA |
| 13 | PT30342 | R | AATGCAAGACCCTGTCAACC |
| 13 | PT30420 | F | AACAAACCGCTTTCCATTCT |
| 13 | PT30420 | R | GAATTAGGCGCTTTGGGAAT |
| 14a | PT30175 | F | TTAGGCGGCGGTATTCTTAT |
| 14a | PT30175 | R | TATGCCTCAATCCCTTACGC |
| 15 | PT30463 | F | AAGCTGCCCTAGCTCAATCA |
| 15 | PT30463 | R | AACATCACCATTTCCACAAGTTT |
| 16 | PT30412 | F | CATTTAGCCGGGAACATTCA |
| 16 | PT30412 | R | CATGGGATACACACGCAAAG |
| 17 | PT30274 | F | TGACAGCTAAGCTAATAACAGTAAATG |
| 17 | PT30274 | R | GGACTTTGGAGTGTCAAATGC |
| 18 | PT30111 | F | AGCCAGCCACCAAATTTATC |
| 18 | PT30111 | R | GGAACATTGCTCAAGCCCTA |
| 19 | PT30230 | F | TTTCTTTCTGTCTGATGCTTCAAT |
| 19 | PT30230 | R | TTGTCCATCTCACTTGCTGC |
| 20 | PT20286 | F | ACGCTAGAGCATCCAACA |
| 20 | PT20286 | R | TAGTGAAAGGCAAGCAGG |
| 21 | PT30378 | F | TCAAATGAGGGTTGTAGCCA |
| 21 | PT30378 | R | TGCAATGGCTACACAAGAAGA |
| 22 | PT30168 | F | TTGAACACCAATTGCGGTAA |
| 22 | PT30168 | R | AAATTCTTGGGTCATGGTGG |
| 23 | PT30231 | F | AGGAGGCGAAGAAAGAGGAG |
| 23 | PT30231 | R | CCCATGAATTCGTAACAGCA |
| 24 | PT40024 | F | AATGTCTGCCCAATCGAAAG |
| 24 | PT40024 | R | CGAATAACGACACTCGAACG |

### EXAMPLE 1

### Transmission of ptDNA in N. tabacum/N. sylvestris Graft Tissue

Our objective was to determine if chloroplasts or mitochondria could be shared among supracellular plant cells. To test this hypothesis, we grafted two different species of tobacco with genetic markers in their plastids and mitochondria. Grafting triggers formation of new plasmodesmatal connections (9) that creates a conduit for cell-to cell movement of organelles. We report here evidence supporting the transfer of plastids via newly formed plasmodesmata. However, the related (non-selected) mitochondria were absent in the same plants, suggesting independent transfer of plastids through the graft junction. We discuss acquisition of plastids from neighboring cells via plasmodesmata as a potential mechanism to repopulate cells with functional organelles and new opportunities created by the cell-to-cell movement of plastids for biotechnological applications.

### Experimental Design.

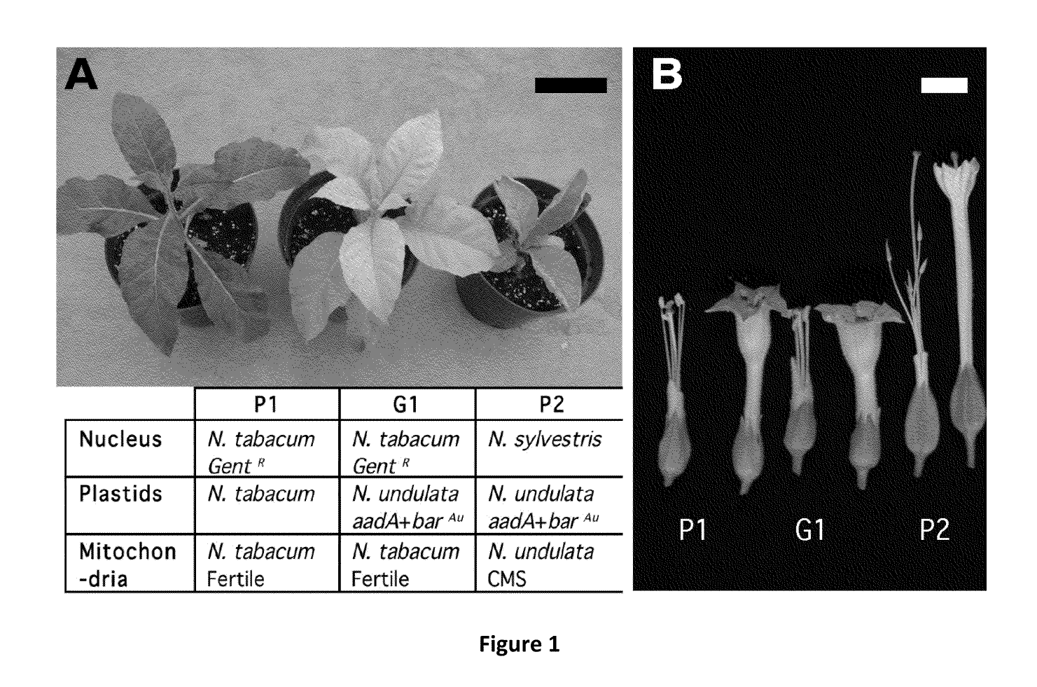

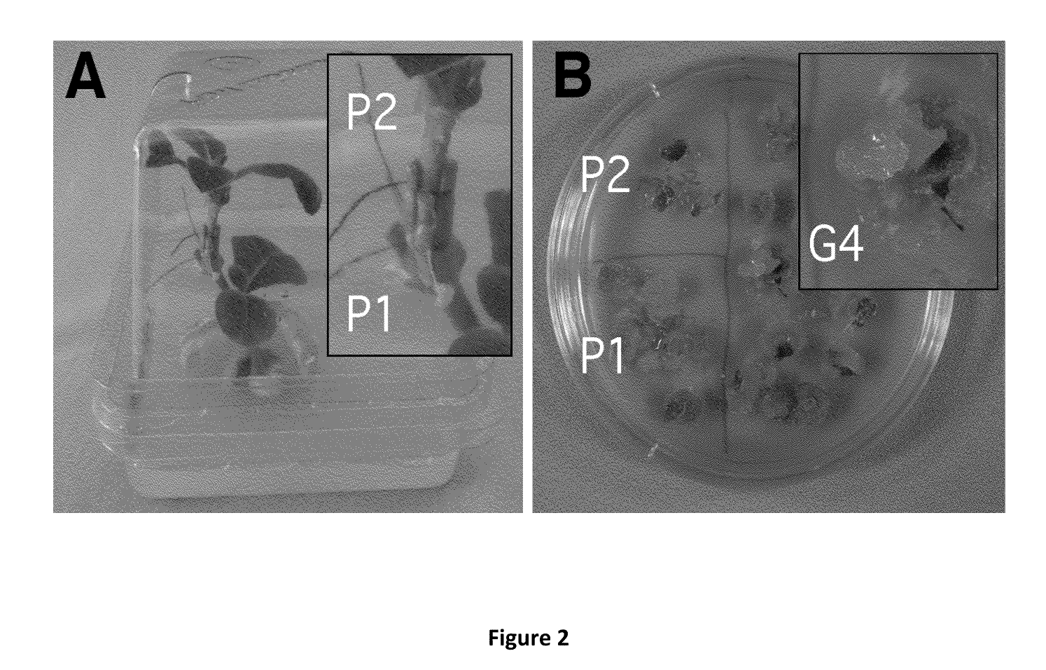

Because of the difficulty to directly observe rare intercellular organelle movement, we chose graft partners with distinct nuclear and organellar genomes to test for cell-to-cell transfer of plastids and mitochondria in graft junctions (FIG. 1). We grafted two species of tobacco, Nicotiana tabacum (partner P1) with a selectable transgenic nuclear gentamycin resistance gene and Nicotiana sylvestris (partner P2) with plastids carrying a selectable spectinomycin resistance (aadA) gene and the aurea young leaf color phenotype (bar^{au} gene). The N. sylvestris partner carried the plastids and mitochondria of a third species, Nicotiana undulata, providing a large number of organellar DNA markers. The P1 partner with the N. tabacum nucleus was fertile and the P2 partner with the N. sylvestris nucleus cytoplasmic male sterile (CMS) (FIG. 1B), a trait controlled by mitochondria (16). The grafted plants were grown in culture for ten days (FIG. 2A) and sections of the graft junctions were selected for the gentamycin and spectinomycin resistance traits carried by the P1 nucleus and in P2 plastids, respectively (FIG. 2B). Out of 30 graft junctions a total of three plastid graft transmission (PGT) events (G1, G3, G4) were recovered. The plants regenerated from the graft junction displayed the leaf morphology, growth habit and pink flowers associated with the selected N. tabacum nucleus, but the aurea leaf color of the P2 partner, a plastid trait (FIGS. 1A and B).

### No Exchange of Chromosomes in the PGT Plants.

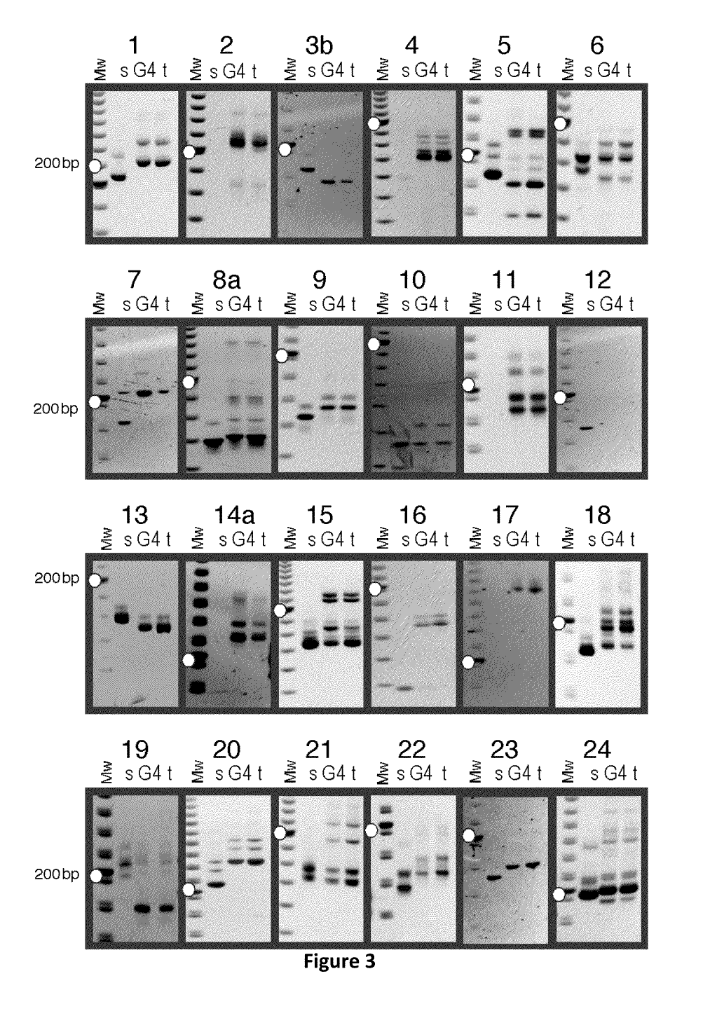

To investigate the contribution of nuclear genetic material to the PGT plants, we examined twenty-four simple sequence repeat (SSR, or microsatellite) polymorphic DNA markers previously mapped to each of the N. tabacum chromosomes (8). These markers distinguished N. tabacum from N. sylvestris ecotype TW137 and indicated the presence of the chromosomes of the N. tabacum P1 partner that carried the selectable nuclear gene without contribution from the non-selected P2 N. sylvestris nucleus (FIG. 3). The presence of chromosomal markers from one partner excluded chimera formation as the source of double resistance of the G1, G3 and G4 PGT plants.

Mitochondria Remain Associated with the Selected Nucleus.

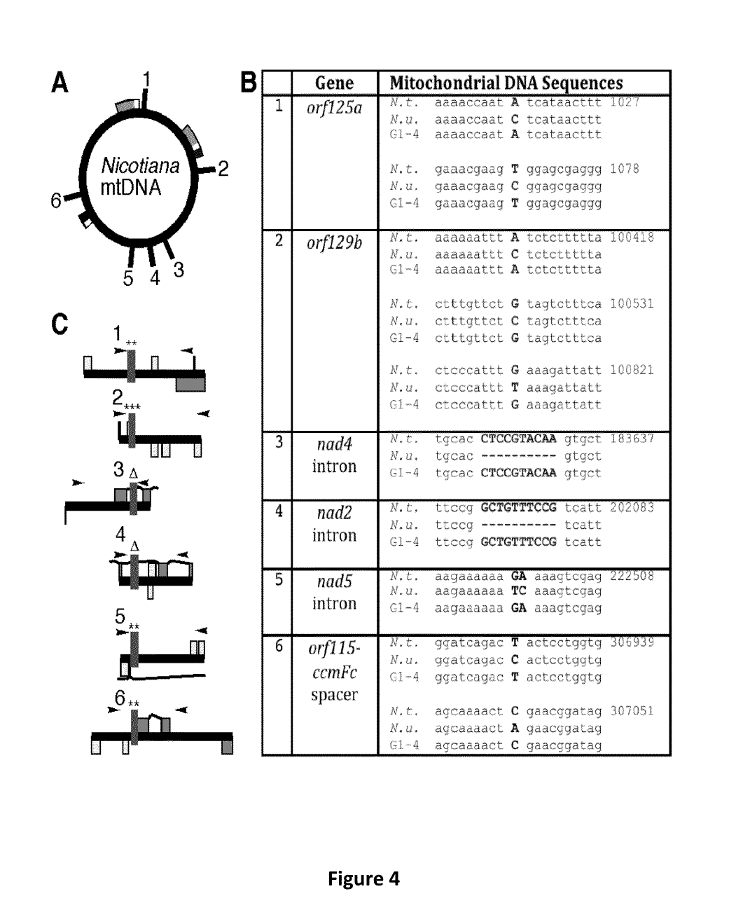

The graft partners carried distinct mitochondrial genomes determining the flower type (FIG. 1B). The P1 partner with the N. tabacum nucleus had normal anthers and produced fertile pollen while the P2 partner with the N. sylvestris nucleus had stigmatoid anthers, a phenotype controlled by mitochondria. Interestingly, the G1, G3 and G4 PGT plants were male fertile and lacked the stigmatoid anthers of the CMS P2 partner. In line with the flower morphology, the CMS92 mtDNA markers were absent in the G1, G3 and G4 plants. To determine the source of the mitochondrial genome in the PGT plants, we identified six SNP and indel markers that are suitable to distinguish the N. undulata CMS92 mtDNA (FIG. 4) from the fertile N. tabacum mtDNA (17). Sanger sequencing of PCR fragments indicated that the G1, G3 and G4 plants have the mitochondrial genome of the nuclear donor (FIG. 4). Thus, we did not find evidence for the transfer of mitochondrial DNA in the PGT plants. Given the tendency of mitochondria for fusion (18) and mtDNA for recombination (16, 19), would mtDNA be transferred, we would expect to find at least chimeric mtDNA. The absence of non-selected mitochondrial DNA suggests limited organelle transfer that did not involve large-scale mixing of the two cytoplasms at the graft junction. Although we did not find evidence for the co-transfer of mtDNA transfer with ptDNA in the lines tested, it is possible that mtDNA transfer could be detected in a larger PGT plant population.

### PGT Plants Contain the Entire Selected Plastid Genome.

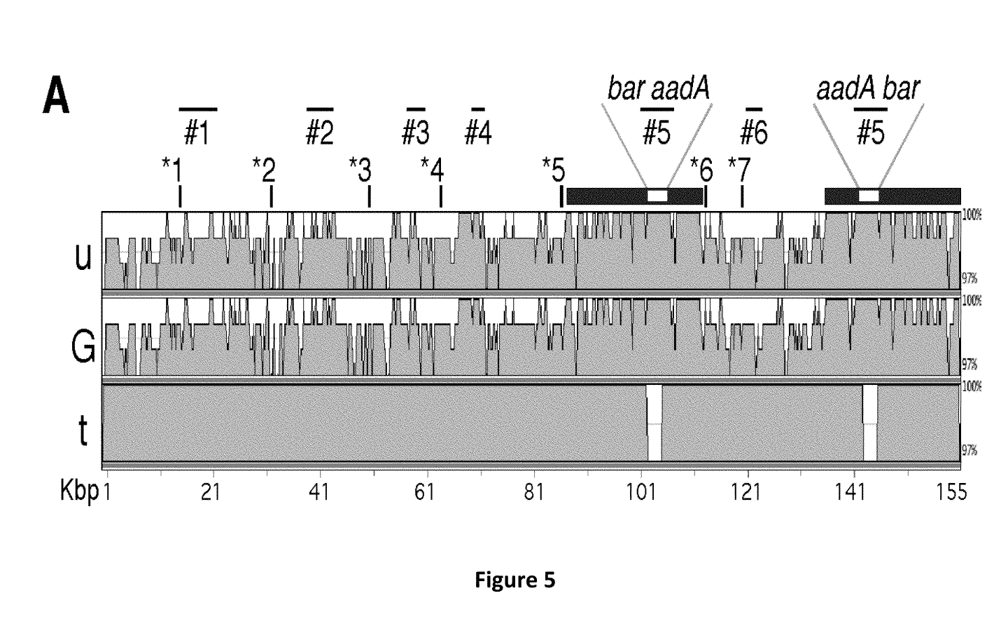

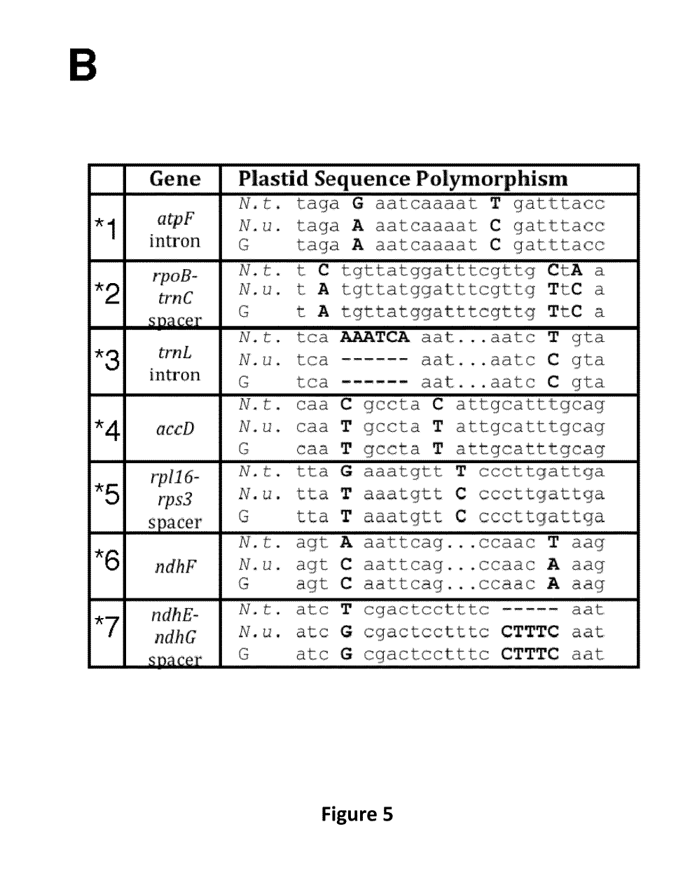

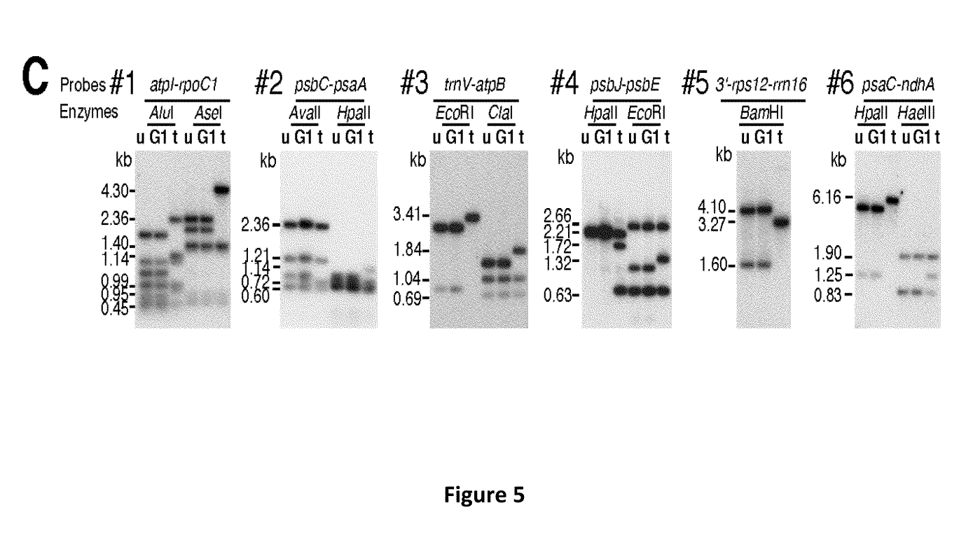

Dual selection for the nucleus- and plastid-encoded antibiotic resistances ensured that the PGT plants would carry both transgenes. The N. tabacum-specific SSR markers in the G1, G3 and G4 plants indicated the presence of the P1 chromosomes alone in the PGT plants. However, the presence of the plastid markers did not distinguish between a transformation-like process that involves incorporation of ptDNA fragments and intercellular movement of plastids implied by the transfer of complete plastid genomes, either of which is compatible with the earlier report (4). To determine how much of the P2 ptDNA is present in the G1, G3 and G4 plants, we first examined markers distant from the transgenes by probing total cellular DNA on blots. Southern probing of the six previously identified RFLP markers (FIG. 5C) and PCR analyses (FIG. 5B) suggested the presence of the entire plastid genome of P2 partner and that the PGT plants carried a uniform population of P2 transplastomes. To exhaust the search for a contribution to the G1 plastid genome from the non-selected P1 plastome, we performed next generation sequencing of the plastid genomes of the P1 and P2 partners and the G1, G3 and G4 PGT plants. We report here the sequence of the 160,743 nucleotide transplastomes in the P2 partner and the three PGT plants are identical (GenBank accession no. JN563930). The P2 and PGT plastid genomes are larger than the 155,863 nucleotide wild type N. undulata plastid genome (GenBank accession no. JN563929), as the transplastomes also contain spectinomycin resistance (aadA) and the aurea bar^{au} transgenes. We also sequenced the plastid genome in partner P1 that carries the wild-type N. tabacum ptDNA of cv. Petit Havana. We have found that the sequence of cv. Petit Havana ptDNA is identical with the cv. Bright Yellow sequence deposited in GenBank (GenBank Accession number Z00044). However, the N. undulata ptDNA differs from the N. tabacum cv. Petit Havana ptDNA by 805 SNPs, 52 insertions and 61 deletions and the transgene cassettes. Differences between the plastid genomes are depicted on the mVISTA identity plots shown in FIG. 5A. Importantly, we observed all of these polymorphic loci, with an average density of 200 bp/SNP (170 bp/polymorphism) in the PGT graft transmission plants indicating the transfer of intact ptDNA from the P2 graft partner. We also tested transmission of the plastid-encoded spectinomycin resistance in reciprocal backcrosses. When the G1 plant was the mother and the wild type the father, each of the 208 seedlings was resistant whereas when the G1 plant was the father and the wild type the mother, each of the 318 seedlings was spectinomycin sensitive. Thus, spectinomycin resistance exhibited uniform, maternal inheritance, as expected for a homoplastomic N. tabacum, a species with strict maternal plastid inheritance.

### Cell-to-Cell Migration of Plastids.

We report here cell-to-cell movement of entire plastid genomes. We considered two possible mechanisms for the transfer of genome-size ptDNA: the intercellular transport of extra-organellar (“naked”) DNA or the ptDNA traveling within an intact organelle. Selection for movement of ptDNA to the nucleus lead to the discovery of ptDNA transfer to the nucleus by incorporation of kilobase-size ptDNA fragments, most probably from degraded organellar genomes (20-22). Movement of entire genomes may require more protection than the fragments. Better protection could be provided if the extra-organellar ptDNA would be encapsulated in membrane-bound vesicles that are shed from fragmented chloroplast stromules (23). Because of the need for capacity for translation, plastids cannot be created de novo from membranes and DNA (24). Thus, if “naked” ptDNA is transferred, an invading plastome would need to enter an existing plastid with transcription and translation machinery and displace the existing plastome by a transformation-like process to explain our observations. However, a transformation-like process would yield mosaic genomes if different genomes were present, because plastid genomes within an organelle undergo frequent recombination (25-27). The absence of chimeric genomes in the PGT plants makes it unlikely that naked DNA transfer is the mechanism of intercellular ptDNA transfer.

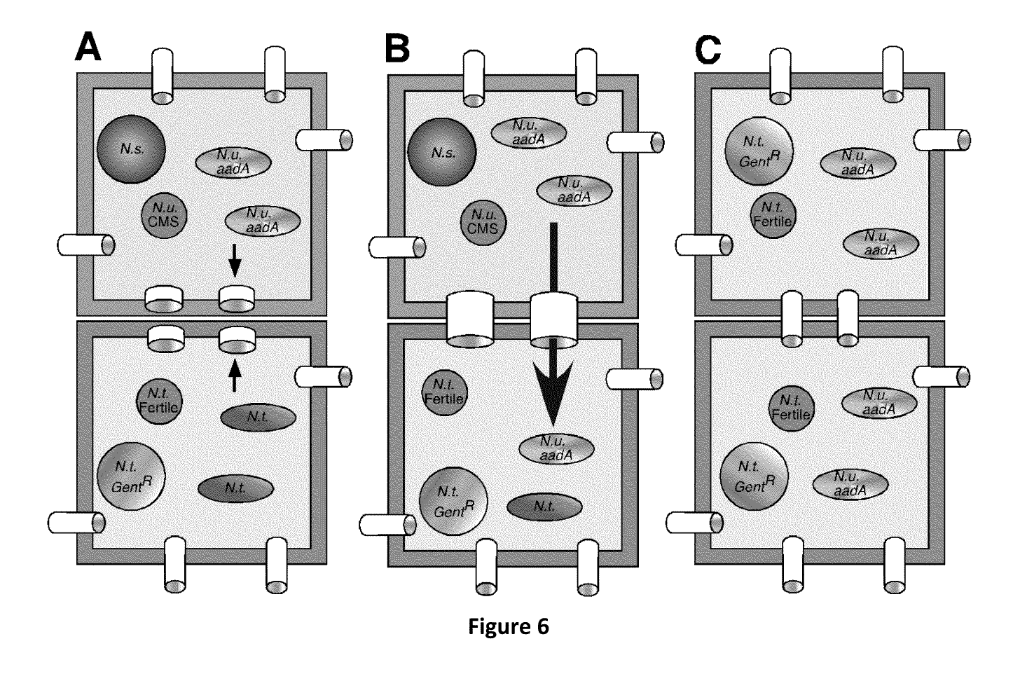

More likely vehicles of cell-to-cell movement of entire plastid genomes could be the organelles themselves. The avenue for the movement of intact organelles could be damage to cell walls that allows for some mixing of cytoplasms in the graft junctions. A more likely mechanism would be the transfer of proplastids via newly formed connections between cells that are well documented at graft junctions (9). The size of proplastids, about one micrometer, is well above the size exclusion limit of plasmodesmata normally defined by molecular weight. However, the size exclusion limit changes during development and depends on tissue type (1, 28). We speculate that the new openings, formed by thinning of opposing cell walls at the site of future plasmodesmata, permit intercellular movement of proplastids. Our preferred model of intercellular plastid transfer in graft junctions is shown in FIG. 6.

The capacity of a plant cell to acquire organelles from a neighboring cell is a basic biological process. Acquisition of plastids from neighboring cells may be important because once the ribosomes are lost, translation cannot be restored, since some of the ribosomal proteins are encoded in the plastid genome and their translation is dependent on plastid ribosomes (24). Therefore, during certain stages of development, including dedifferentiation associated with forming new connections in grafted tissues (9), the plasmodesmata may allow the transport of organelles to ensure the continuity of functional DNA containing organelles. In this regard it is intriguing to note that the redox state of plastids regulates symplastic permeability and that ectopic expression of the proplastid-targeted GAT1 protein increased plasmodesmal size exclusion limit (29). The functional state of mitochondria also regulates the size exclusion limit of intercellular trafficking (30) and reprogramming of diseased mammalian cells was associated with acquisition of functional mitochondria (6). The discovery of intercellular movement of plastids now enables testing the biological significance of this process in plants.

While the protocol described here is based on wedge grafting, decapitation of the rootstock and separation of scion from its root system is not necessary to obtain grafting. Natural grafting has been observed between plants in nature, when graft junction forms between plants growing in close proximity (31). Accordingly, wedge grafting may be replaced by alternative protocols based on natural grafting. In one approach, the surface of the stem of the graft partners are removed and the stems are tied together to mimic natural grafts. PGT plants can be recovered from the graft junctions by tissue culture selection as described in the present application, or identified based on plant morphological markers and visual plastid markers in shoots regenerated from the graft junction. See U.S. patent application Ser. No. 13/326,295.

Intercellular movement of organelles should not be limited to intact plants, but should be applicable to any two cells making a new contact enabling cell-to-cell movement of plant organelles. Such cells may be in tissue culture, said first and second plants comprising distinct plastid and nuclear genetic markers, enabling selection for PGT events. Recovery of PGT (organelle) events in tissue culture may be particularly beneficial when grafting is technically challenging, such as in monocotyledonous plants.

### Applications in Plastid Genetics and Biotechnology.

Because in most species both plastids and mitochondria are maternally inherited, they cannot be separated by crossing. Thus far protoplast fusion has been the only option to obtain new combinations of plastids and mitochondria (16). The result is intercellular transfer of parental plastids, but formation of recombinant mitochondrial genomes. The protocol we report here enables combination of parental plastids and non-recombinant mitochondria by PGT, a significant improvement over the protoplast-based process that yields recombinant mitochondria.

An additional application of PGT could be rapid introgression of transformed plastids into commercial cultivars. Plastid transformation is a powerful tool for biotechnological applications because the transgenes that are integrated into the plastid genome are expressed at high levels, can be clustered in operons and are not subject to silencing (32, 33). Currently the option is to transform the plastids in permissive cultivars then introduce them into commercial lines by repeated backcrossing using the commercial cultivar as a recurrent pollen parent. Based on the findings disclosed herein, backcrossing can be replaced in the future by graft transfer of the transformed plastids, instantly yielding a substitution line carrying the valuable commercial nuclear genome combined with transgenic plastids.

### EXAMPLE 2

### Introduction of Autoluminescent Chloroplasts into Genetically Sterile Plants or into Plants Lacking Flowers

Plastid transformation currently is a tissue culture dependent protocol that can be performed only with tissue-culture responsive genetic lines. Introduction of transformed plastid genomes into commercially useful lines requires repeated cycles of backcrosses. Inter-cellular transfer of organellar DNA in tissue grafts enables one-step transfer of plastid genomes in the absence of the transfer of nuclear genetic information, eliminating the need for backcrosses. Furthermore, graft transfer of plastids is possible between sterile plants lacking flowers and between sexually incompatible genetic lines.

Desirable plastids for transfer by non-sexual means may be autoluminescent plastids of different plant species carrying the lux operon (34) and the following recipients:
(1) Fertile lines that are sexually compatible, but encode desirable traits in their nuclei.
(2) Fertile lines that are sexually incompatible, thus introduction could not be accomplished by crossing.
(3) Plants, which lack flower organs or have flower organs but are sterile.

### EXAMPLE 3

### Transmission of mtDNA in the N. tabacum/N. sylvestris Graft

We did not find evidence for co-transfer of the non-selected mtDNA with the selected ptDNA. Even if the mitochondria (mtDNA) were co-transferred with plastids, they were likely lost due to the absence of direct selection for mitochondrial traits. Thus, testing a larger population of PGT plants could possibly yield plants expressing the CMS flower morphology, a mitochondrial trait. A factor in the lack of recovering CMS plants could be the presumed recessive nature of Nicotiana undulata CMS, implied by the relatively small number of CMS plants recovered in somatic hybrids (35). Because in our case plastids from the CMS P2 partner have moved into the fertile P1 partner, if recessive, the CMS mitochondrial trait remains undetected, unless the dominant fertile mitochondrial determinants are lost. In order to increase the likelihood of detecting the co-transfer of mitochondria (mtDNA) with plastids, we will utilize fertile plants as the source of plastids, because detecting restoration of fertile flower morphology, a dominant trait, in a sterile partner is more likely in regenerated plants.

It is clear that the foregoing methods are useful for engineering plants and crops having desirable characteristics without the need for extensive back crossing.

### REFERENCES

1. Lucas W J, Ham B K, & Kim J Y (2009) Plasmodesmata—bridging the gap between neighboring plant cells. Trends Cell Biol 19:495-503.
2. Rustom A, Saffrich R, Markovic I, Walther P, & Gerdes H H (2004) Nanotubular highways for intercellular organelle transport. Science 303:1007-1010.
3. Molnar A, et al. (2010) Small silencing RNAs in plants are mobile and direct epigenetic modification in recipient cells. Science 328:872-875.
4. Stegemann S & Bock R (2009) Exchange of genetic material between cells in plant tissue grafts. Science 324:649-651.
5. Koyanagi M, Brandes R P, Haendeler J, Zeiher A M, & Dimmeler S (2005) Cell-to-cell connection of endothelial progenitor cells with cardiac myocytes by nanotubes: a novel mechanism for cell fate changes? Circ Res 96:1039-1041.
6. Acquistapace A, et al. (2011) Human mesenchymal stem cells reprogram adult cardiomyocytes toward a progenitor-like state through partial cell fusion and mitochondria transfer. Stem Cells 29:812-824.
7. Dubey G P & Ben-Yehuda S (2011) Intercellular nanotubes mediate bacterial communication. Cell 144:590-600.
8. Moon H S, Nicholson J S, & Lewis R S (2008) Use of transferable Nicotiana tabacum L. microsatellite markers for investigating genetic diversity in the genus Nicotiana. Genome 51:547-559.
9. Ehlers K & Kollmann R (2001) Primary and secondary plasmodesmata: structure, origin, and functioning. Protoplasma 216:1-30.
10. Carrer H, Staub J M, & Maliga P (1990) Gentamycin resistance in Nicotiana conferred by AAC(3)-I, a narrow substrate specificity acetyl transferase. Plant Mol Biol 17 :301-303.
11. Maliga P & Svab Z (2011) Engineering the plastid genome of Nicotiana sylvestris, a diploid model species for plastid genetics Plant chromosome engineering: methods and protocols, ed Birchler J J (Springer Science+Business Media, LLC, New York), Vol 701, pp 37-50.
12. Lutz K A, Svab Z, & Maliga P (2006) Construction of marker-free transplastomic tobacco using the Cre-loxP site-specific recombination system. Nat Protocols 1:900-910.
13. Svab Z & Maliga P (2007) Exceptional transmission of plastids and mitochondria from the transplastomic pollen parent and its impact on transgene containment. Proc Natl Acad Sci USA 104:7003-7008.
14. Murray M G & Thompson W F (1980) Rapid isolation of high molecular weight plant DNA. Nucleic Acids Res 8:4321-4325.
15. Cronn R, et al. (2008) Multiplex sequencing of plant chloroplast genomes using Solexa sequencing-by-synthesis technology. Nucleic Acids Res 36:e122.
16. Gillman J D, Bentolila S, & Hanson M R (2009) Cytoplasmic male sterility in Petunia. Petunia, eds Gerats T & Strommer J (Springer Science+Business Media, LLC, New York), pp 107-129.
17. Yukawa M, Tsudzuki T, & Sugiura M (2006) The chloroplast genome of Nicotiana sylvestris and Nicotiana tomentosiformis: complete sequencing confirms that the Nicotiana sylvestris progenitor is the maternal genome donor of Nicotiana tabacum. Mol Genet Genomics 275:367-373.
18. Sheahan M B, McCurdy D W, & Rose R J (2005) Mitochondria as a connected population: ensuring continuity of the mitochondrial genome during plant cell dedifferentiation through massive mitochondrial fusion. Plant J 44:744-755.
19. Boeshore M L, Hanson M R, & Izhar S (1985) A variant mitochondrial DNA arrangement specific to Petunia stable sterile somatic hybrids. Plant Mol Biol 4:125-132.
20. Huang C Y, Ayliffe M A, & Timmis J N (2003) Direct measurement of the transfer rate of chloroplast DNA into the nucleus. Nature 422:72-76.
21. Stegemann S, Hartmann S, Ruf S, & Bock R (2003) High-frequency gene transfer from the chloroplast genome to the nucleus. Proc Natl Acad Sci USA 100:8828-8833.
22. Sheppard A E, et al. (2008) Transfer of plastid DNA to the nucleus is elevated during male gametogenesis in tobacco. Plant Physiol 148:328-336.
23. Hanson M R & Sattarzadeh A (2011) Stromules: recent insights into a long neglected feature of plastid morphology and function. Plant Physiol 155:1486-1492.
24. Zubko M K & Day A (1998) Stable albinism induced without mutagenesis: a model for ribosome-free plastid inheritance. Plant J 15:265-271.
25. Palmer J D (1983) Chloroplast DNA exists in two orientations. Nature 301:92-93.
26. Medgyesy P, Fejes E, & Maliga P (1985) Interspecific chloroplast recombination in a Nicotiana somatic hybrid. Proc Natl Acad Sci USA 82:6960-6964.
27. Fejes E, Engler D, & Maliga P (1990) Extensive homologous chloroplast DNA recombination in the pt14 Nicotiana somatic hybrid. Theor Appl Genet 79:28-32.
28. Burch-Smith T M, Stonebloom S, Xu M, & Zambryski P C (2011) Plasmodesmata during development: re-examination of the importance of primary, secondary, and branched plasmodesmata structure versus function. Protoplasma 248:61-74.
29. Benitez-Alfonso Y, et al. (2009) Control of Arabidopsis meristem development by thioredoxin-dependent regulation of intercellular transport. Proc Natl Acad Sci USA 106:3615-3620.
30. Stonebloom S, et al. (2009) Loss of the plant DEAD-box protein ISE1 leads to defective mitochondria and increased cell-to-cell transport via plasmodesmata. Proc Natl Acad Sci USA 106:17229-17234.
31. Bock R (2010) The give-and-take of DNA: horizontal gene transfer in plants. Trends Plant Sci 15:11-22.
32. Maliga P & Bock R (2011) Plastid biotechnology: food, fuel and medicine for the 21st century. Plant Physiol 155:1501-1510.
33. Cardi T, Lenzi P, & Maliga P (2010) Chloroplasts as expression platforms for plant-produced vaccines. Expert Rev Vaccines 9:893-911.
34. Krichevsky A, Meyers B, Vainstein A, Maliga P, & Citovsky V (2010) Autoluminescent plants. PLoS One 5:e15461.
35. Bonnett H T & Glimelius K (1983) Somatic hybridization in Nicotiana: behaviour of organelles after fusion of protoplasts from male-fertile and male-sterile cultivars. Theor Appl Genet 65:213-217.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for effecting intercellular transfer of organelles in plants for the creation of transgenic plants exhibiting desirable characteristics comprising:
a) joining the cells of the first plant and a second plant, said first and second plants comprising distinct plastid and nuclear genetic markers; or
b) joining a first plant and a second plant, said first and second plants comprising distinct plastid and nuclear genetic markers; or
c) joining a root stock of a first plant and a scion from a second plant, said first and second plants comprising distinct plastid and nuclear genetic markers; and
d) culturing said plants for a suitable period for grafting to occur;
i) fragmenting or slicing the graft region and
ii) transferring said fragment or slice to a plant regeneration medium and selecting for cells expressing the nuclear and plastid genetic markers from said first and second plants; or,
e) forcing shoot formation from the graft junction and
i) identifying plastid gene transfer events by altered plant morphology and/or visually detectable plastid-specific markers.

2. The method of claim 1, further comprising characterization of the size and type of DNA transferred.

3. The method of claim 1, wherein said organelle is a plastid and said method results in complete transfer of the plastid genome.

4. The method of claim 3, wherein said transferred plastid genome comprises at least one heterologous DNA molecule expressing at least one protein of interest.

5. The method of claim 4, wherein said heterologous DNA encodes a protein conferring herbicide or drought resistance.

6. The method of claim 4, wherein said heterologous DNA encodes a protein selected from the group consisting of a fluorescent protein, an antibody, a cytokine, an interferon, a hormone, a selectable marker protein, a coagulation factor and an enzyme or a gene for a small RNA.

7. The method of claim 1, wherein said plant is a solanaceous plant.

8. The method of claim 1, wherein said plant is selected from the group consisting of Nicotiana tabacum, Nicotiana sylvestris, Nicotiana benthamiana, potato, tomato, and eggplant.

9. The method of claim 1, wherein said plant is a dicotyledonous plant.

10. A plant regenerated from the cells from step c) of claim 1.

11. Progeny and seed from the plant of claim 10.
